(19) United States
(12) Patent Application Publication
(10) Pub. No.: US 2024/0050200 A1
(43) Pub. Date: Feb. 15, 2024

(54) APPARATUS AND METHOD FOR ORAL SCANNING

(71) Applicant: MEDICAL TECHNOLOGY INNOVATIONS S.R.L, MILANO (IT)

(72) Inventors: Francesco GALLO, MILANO (IT); Giulio NEGRI, LECCO (IT); Francesco ZINGARI, MILANO (IT)

(73) Assignee: MEDICAL TECHNOLOGY INNOVATIONS S.R.L, MILANO (IT)

(21) Appl. No.: 18/258,259

(22) PCT Filed: Dec. 10, 2021
(86) PCT No.: PCT/EP2021/085249

§ 371 Date: Jun. 19, 2023

(30) Foreign Application Priority Data
Dec. 21, 2020 (IT) 102020000031670

(51) Int. Cl. A61C 8/00; A61B 1/24; A61B 1/00; A61C 9/00

(52) U.S. Cl. CPC A61C 8/0001; A61B 1/24; A61B 1/00172; A61C 9/0053

(57) ABSTRACT

An apparatus for digitally scanning dental prosthetic implants, including a plurality of scanbodies, each surrounded by a thread ring; each thread ring engages a portion of a linking thread; each scanbody is associable with a prosthesis or with the oral cavity. The scanning method includes the steps of: removing the prosthesis from the oral cavity, associating the scanbodies with the prosthesis outside the oral cavity, associating the scanbodies with one another by means of the thread rings and the linking thread, performing a digital scan of the entire unit outside the oral cavity or screwing the scanbodies to each implant inserted in the bone, associating the scanbodies with one another by means of the thread rings and the linking thread, performing a scan in the oral cavity.

The present invention relates to an apparatus and a method for oral scanning, particularly for the digital scanning of dental prosthetic implants.

The traditional impression acquisition technique is a clinical difficulty for the dentist, who, despite being expert, must perform the maneuvers at the chair in short and separate times, depending on the materials used.

Patient compliance modifies the quality of the clinical gestures and therefore affects the result of the final impression.

The introduction of digital techniques in the acquisition of impressions, by means of intraoral scanners, has significantly reduced the difficulties described above, rendering impression acquisition more predictable and reliable for the dentist and more comfortable for the patient, allowing execution times which are not conditioned by the impression materials and are in any case quite short.

US2014/124374A1 discloses a position locator for use in dental restorative procedure. The position locator is inserted into a replica of a dental implant or into a replica of an abutment. The position and orientation of the implant replica is determined by scanning the model with the implant replica and the position locator. Alternatively, the position locator can be inserted into the dental implant and scanning is carried out in the mouth of the patient. The position locator is made of an optically opaque material, such as titanium, and has an outer surface detectable by an optical scanner, e.g. with a layer of porous titanium oxide applied through anodic oxidation.

US2016/015488A1 discloses a precalibrated dental implant kit comprising a visualizer for a dental implant which comprises a mechanical connection part to a predetermined implant root defining, relative to the visualizer, the vector of the implant root along the axis of orientation, and three markers, contrasting under electromagnetic radiation with their surroundings. The markers being observable and recognizable by an electromagnetic recognition technique. The markers define a geometric pattern of which the recognition technique is capable of collecting the spatial information. A data file from which the spatial information of the geometric pattern is retrieved and referenced thereto information determining the vector of the implant root relative to the pattern, and also identifying the visualizer and the predetermined implant root.

US2018/206951A1 discloses a method for designing and manufacturing implant based restorations involving the use of Scannable Temporary Anatomic References that should provide reliable, fixed points of reference to enable a technician to relate and superimpose the dental implant position to a pre-planned restoration, with a degree of accuracy throughout the entire workflow. The method can be carried out by placing Scannable Temporary Anatomic References in a jaw, on teeth, and/or in implants, collecting images by scanning a mouth, relating those images to images of an appearance of desired dental implant based restoration, placing an implant in the jaw, scanning the scannable temporary anatomic references to create a new set of images, relating the images, and producing a restoration based on the images.

Basically, digital scanners project a light source onto an object that returns information which is converted into three-dimensional images.

Currently, in order to acquire digital impressions on single implants or multiple implants, known elements with known geometries, already included in a library, are used and in most cases are screwed to the implants themselves.

The light source strikes those known elements and captures their position in the three Cartesian axes, essentially acquiring the position of the implant in terms of depth, inclination and relationship with the other implants or natural teeth.

These known elements are generally known as scanbodies.

The implant manufacturers produce their own scanbody with different but generally standardized shapes, made of PEK or titanium, which can be cold-processed in an autoclave or hot-processed in a sterilizer, with a metal connection, in 90% of cases. Therefore, each scanbody is specific to its implant and cannot be modified in its known geometric shape.

Digital software acquires this known geometric shape and converts the image into STL files, which can then be processed in CAD/CAM in order to produce models with 3D printers or directly artificial teeth.

The STL files that digital scanners acquire are read by CAD/CAM systems and, before producing the artificial tooth or teeth, the operator checks that the acquired impression of the scanbody is precise.

This operation, performed by the software, is known as “best fit” and essentially consists in superimposing two images (files), the image of the scanbody acquired with an optical impression and the one of the scanbody contained in the virtual library of the CAD/CAM systems.

Only when the match between the scanbody acquired in the oral cavity and the scanbody of the virtual library is correct, ensuring the precision of the scan, the artificial tooth or teeth can be produced.

Best fit, i.e., the match between the scanbody acquired in the oral cavity and the library scanbody, is expressed by the CAD/CAM software by means of a color code or by means of a number expressed in microns.

Best fit is an assurance of precision of the scan of the scanbody. Therefore, if the best fit corresponds to a color or a number that are deemed valid, the position of the implant in the bone is known and therefore precise.

Scanning Strategy of Intraoral Scanners

As is known, each currently commercially available scanner has its own scanning strategy which renders the scan precise.

What all scanners have in common is that the scan begins in a specific point, for example A, and ends in another point, termed for example D. Therefore the flow of the scan must proceed from A toward D.

If the scan flow is interrupted, which can be done without problems for the scan itself, it is necessary to resume the scan from a known point along the path that has already been traced.

For example, if in the path from a point A to a point D, given the consecutive points A, B, C, D, the scan is interrupted in point B, when the scan resumes one cannot begin from point C or D but must one must resume the scan from a known point that has already been recorded between points A and B.

Problem of Intraoral Scanners

It is known that reading precision problems can arise, when a digital impression is acquired in a patient who has two or more implants, therefore when two or more scanbodies must be placed in mutual relationship.

While scanbodies are in any case read in their precise measurements, their relationship might in fact not be equally precise. Therefore, if it becomes necessary to create a fixed and joined structure between two or more implants, in order to create for example a bridge of artificial teeth, starting from the scan, the scan might not have the minimum precision standards.

The path that the light source follows between one scanbody and the other should allow the recording of a quantity of files which link each other, without having the slightest distortion.

The problem is that during the image reading or rendering steps performed by the software the files are likely to undergo modifications and “adjustments”.

These modifications produce tolerable errors if they are performed on the individual scanbodies, but produce more important errors if performed between one scanbody and the other, since they modify their actual distance.

Therefore, if the operator must produce a rigid structure between two or more implants, the structure runs the risk of being inaccurate.

A fully empirical system adopted by the dental community to obviate these drawbacks includes positioning, between one scanbody and the other, dummy elements, glued to the mucosa, which do not lose the fluidity and consequentiality of the reading of the scanner; in this manner the files would have no scanning holes.

The critical points of the path of the light source would appear to be:
the inter-implant distance,
the natural curvature of the oral cavity,
the identical geometry of the scanbodies in the case of full-arch or prostheses of the Toronto type.

The greater the distance that the light source must cover between one scanbody and the other, the higher the possibility of scanning error. In this case, many more files must be linked between one scanbody and the other.

As regards the natural curvature of the oral cavity, the most critical point expressed by the dental community resides in that both dental arches have a natural curvature; indeed this anatomical characteristic does not allow the correct linking of the files along the curvature during the path of the light source between one scanbody and the other.

As regards the identical geometry of the scanbodies, in the case of full-arch or prosthesis of the Toronto type, during the reading of multiple scanbodies, from four upward, in totally edentulous patients, in which multiple implants are inserted in order to completely rehabilitate an arch, three problems combine:
the inter-implant distance, which is peculiar to each patient (variable and unknown distance);
the natural curvature of the arch, which is peculiar to each patient (variable and unknown curvature);
the loss of references by the scanner; the scanner confuses the scanbodies of one side with those of the other side, since they are all mutually identical and since the gum mucosa portions between one scanbody and the other, despite being different in all of its points, is unable to render differently one side with respect to the other.

The aim of the present invention is to provide an apparatus and a method for oral and extraoral scanning that overcome the drawbacks of the cited prior art.

Within the scope of this aim, an object of the invention is to provide an apparatus that overcomes the limitations of the digital technique known so far, particularly with regard to the scanning difficulties that are observed in patients with long edentulous saddles (intercalated edentulisms), or in the case of fully edentulous patients with implants.

In the traditional technique, the spaces between one scanbody and the next compromise the reading of the intraoral scanner as they increase. The reading jump that the scanner must perform in these cases is not only sagittal, since it has to pass from one scanbody to the next, but also axial, since it has to pass from the maximum height of the apical part of the scanbody to the minimum one represented by the oral mucosa.

In addition to creating difficulties and slowing the reading of the scanner, these conditions add stitching (deformation) problems during the rendering steps (graphic output).

Another object of the invention is to overcome the problem due to the scanbodies being standard and identical for all implants, creating further difficulties by confusing the software of the scanner, which can even superimpose the scanbodies of one side on those of the other, invalidating the result of the scan.

A further object of the invention is to overcome the problem of traditional scanners caused by the temporary removal of the provisional implant, which is necessary to scan the arches in order to prepare the final prosthesis.

The time required to remove the provisional implant, to assemble the prosthetic components for scanning and for the scan itself can in fact cause a change in the shape of the gum parables due to the natural elasticity of gum tissues. Therefore, the consequent reading of the mucosa performed by the scanner does not precisely correspond to the shape of the provisional implant. The discrepancy between the gum parables reproduced in the provisional implant and those read by the scanner is therefore a prosthetic problem that is transferred unequivocally to the final prosthesis.

This aim and these and other objects which will become better apparent hereinafter are achieved by an apparatus and a method for the digital scanning of dental prosthetic implants, as claimed in the appended claims.

Further characteristics and advantages will become better apparent from the description of preferred but not exclusive embodiments of the invention, illustrated by way of non-limiting example in the accompanying drawings, wherein:

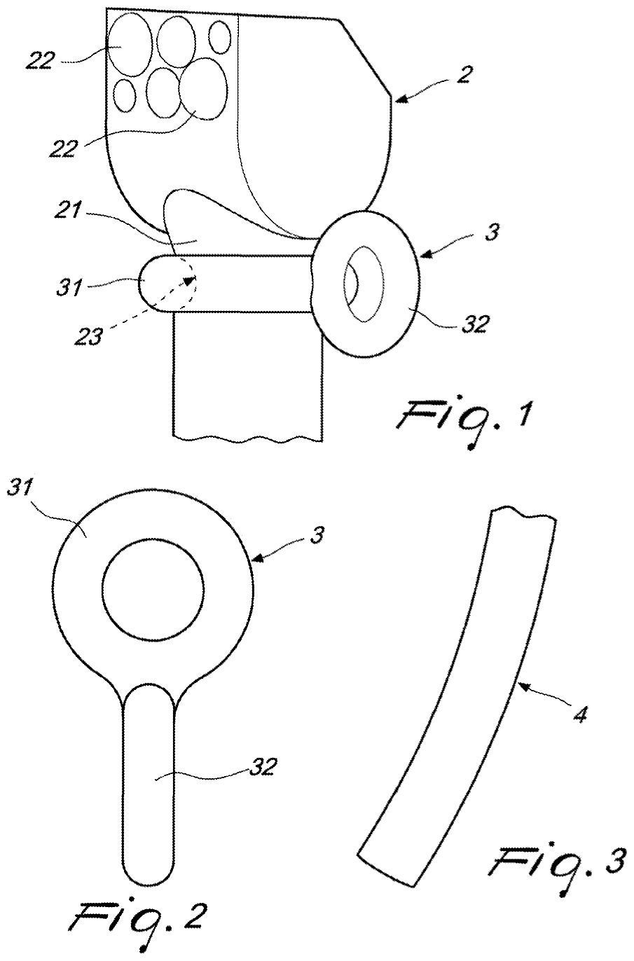

FIG. 1 is a perspective view of a scanbody which is part of the apparatus according to the present invention;

FIG. 2 is a perspective view of a modified figure-of-eight thread ring, which is part of the apparatus according to the present invention;

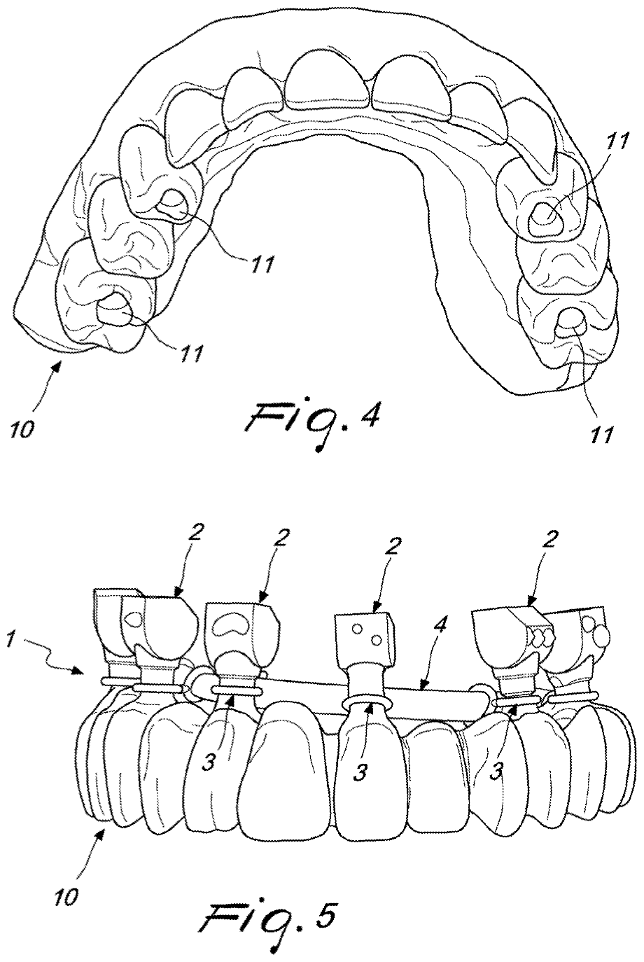

FIG. 3 is a perspective view of a linking thread which is part of the apparatus according to the present invention;

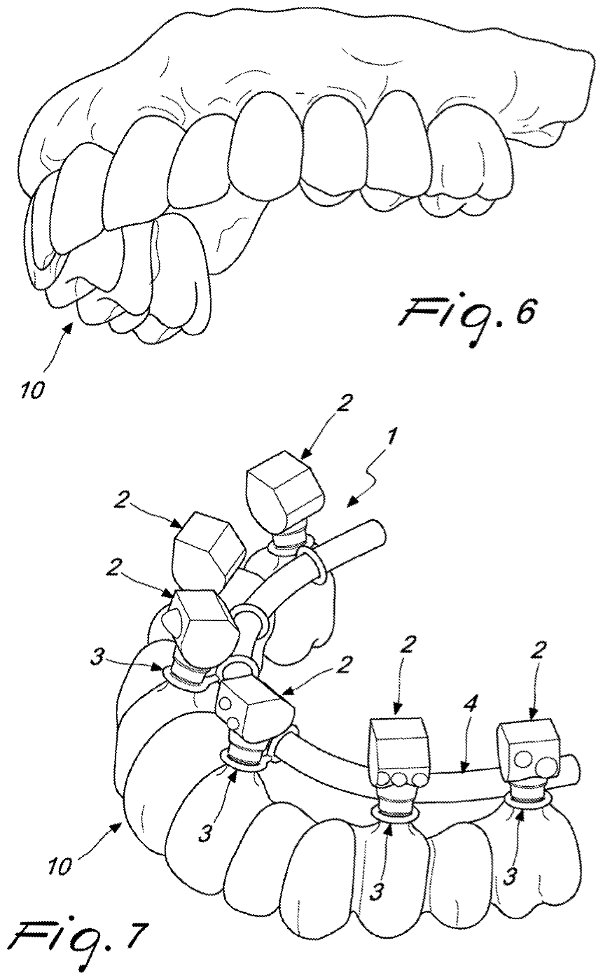

FIGS. 4-7 are perspective views of the use of the apparatus for the reverse scanning of a prosthesis, according to the invention;

FIG. 8 is a side view of an intraoral scanbody;

FIG. 9 is a top view of the scanbody of the preceding figure;

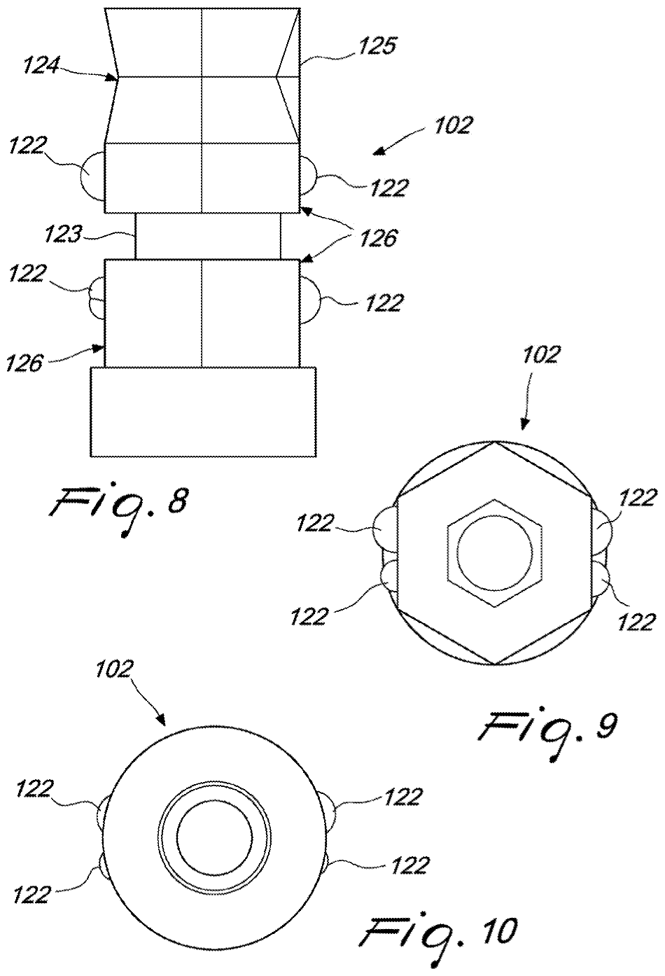

FIG. 10 is a bottom view of the scanbody of the preceding figure;

FIG. 11 is a side view of the scanbody of the preceding figure;

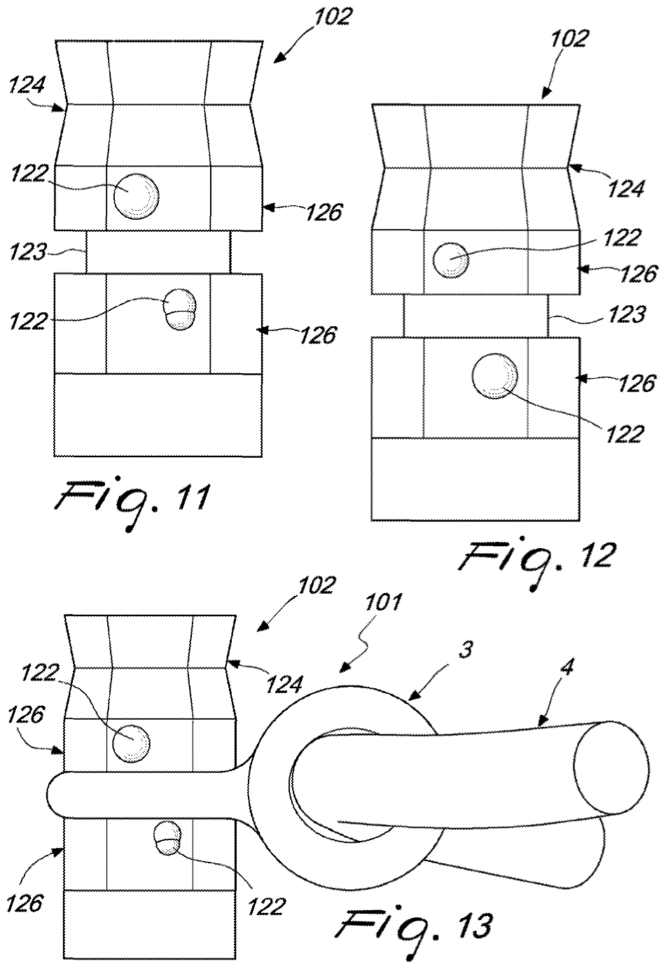

FIG. 12 is a side view of the opposite side of the scanbody of the preceding figure;

FIG. 13 is a side view of the scanbody of the preceding figure, associated with a thread ring and with a portion of a linking thread;

FIG. 14 is a top view of the scanbody of the preceding figure, associated with a thread ring and with a portion of a linking thread;

FIG. 15 is a bottom view of the scanbody of the preceding figure, associated with a thread ring and with a portion of a linking thread;

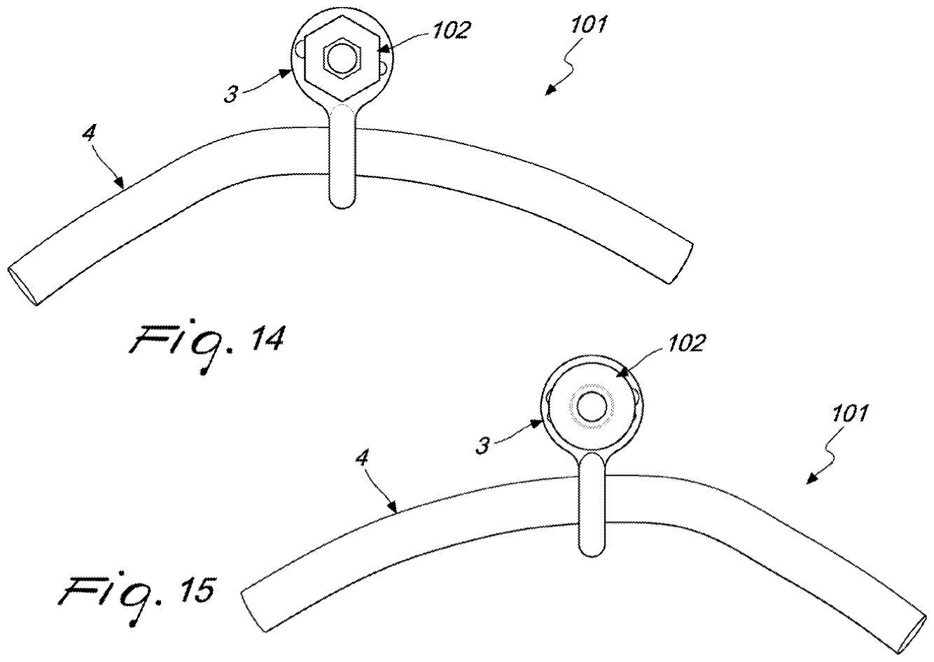

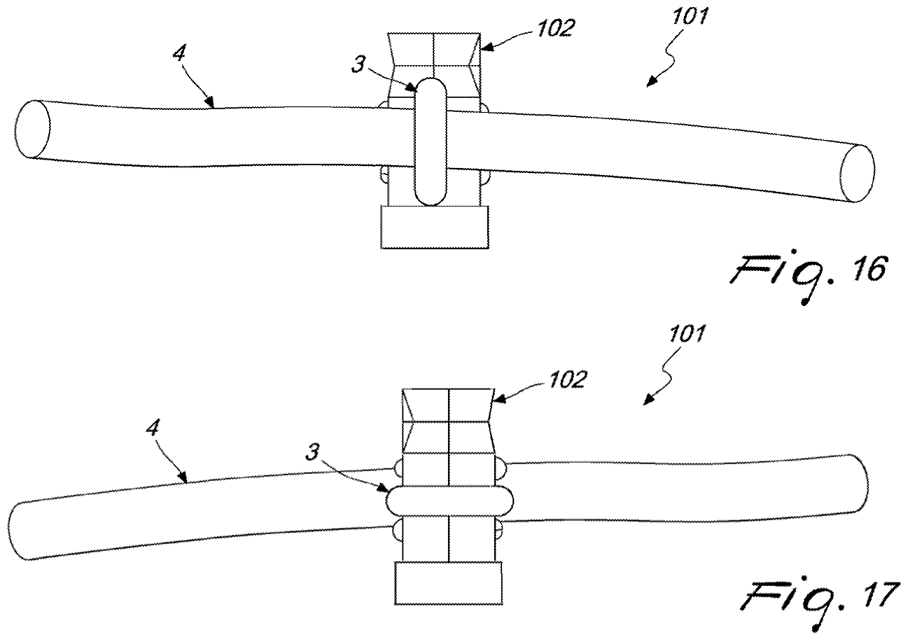

FIG. 16 is a side view of the scanbody of the preceding figure, associated with a thread ring and with a portion of a linking thread;

FIG. 17 is a view, taken from the opposite side with respect to the preceding figure, of the scanbody associated with a thread ring and with a portion of a linking thread;

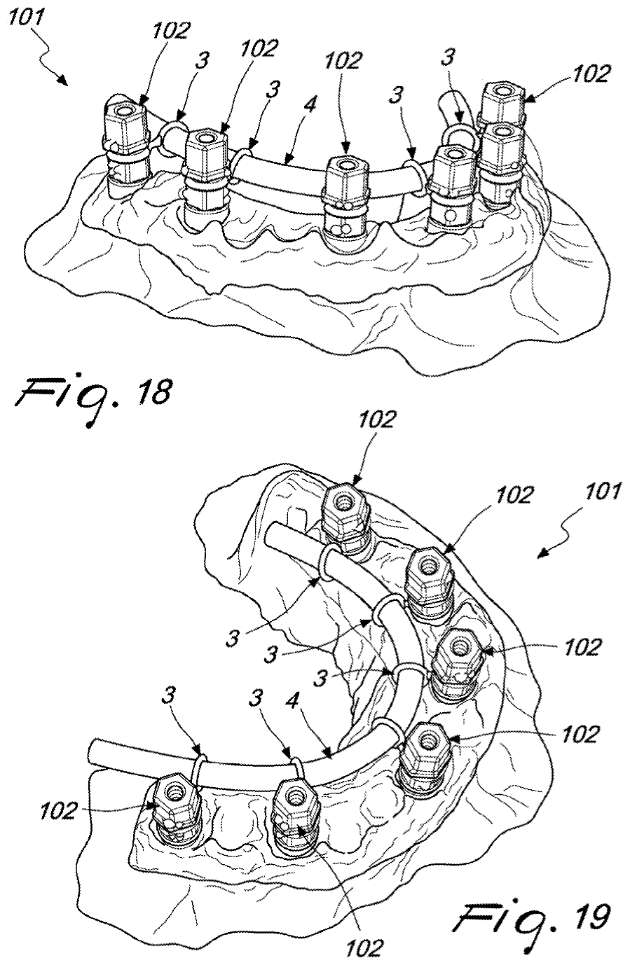

FIGS. 18 and 19 are perspective views of an example of use of the apparatus shown in FIGS. 8-17;

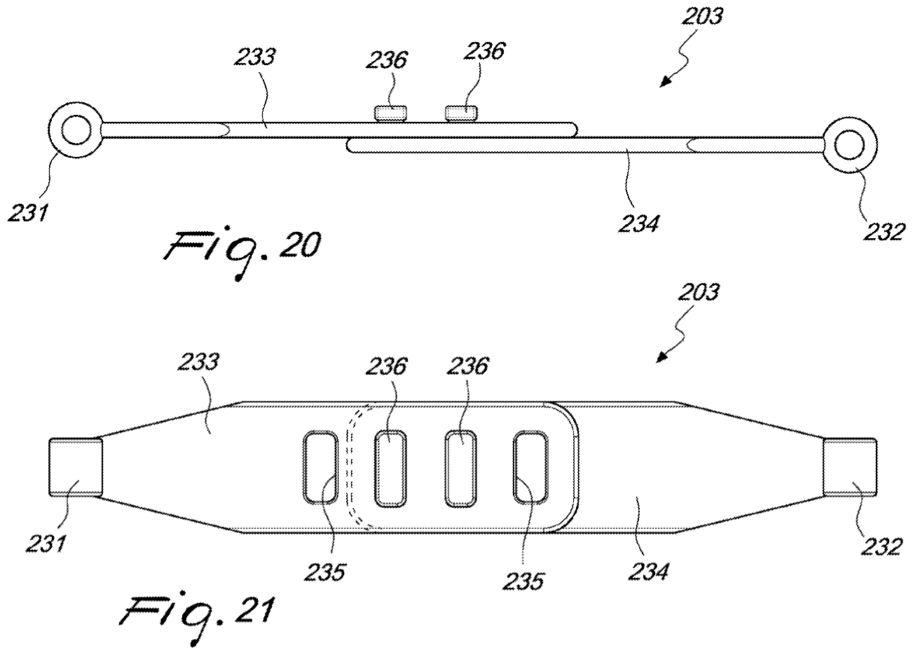

FIG. 20 is a front view of a modified thread ring;

FIG. 21 is a top view of a modified thread ring.

With reference to the cited figures, the apparatus according to the invention, generally designated by the reference numeral 1, has a plurality of scanbodies 2, each surrounded by a ring of elastic thread 3 which in turn engages a portion of a linking thread 4.

The scanbody 2 is a component that has a more or less cubic shape and has a conical or tapering portion, designated by the reference numeral 21, on one side which corresponds to the engagement with a prosthesis 10 to which it is screwed by means of a specific connection 11.

The scanbody 2 is flattened in a vestibular direction in order to facilitate its reading and two of its faces are regular so as to allow them to be recognized by a scanner, during scanning, and to be coupled with the digital libraries.

On the vestibular side, the scanbody 2 has specific facets and protrusions 22 which are aimed at rendering the individual parts unique, preventing the scanner from confusing the various scanbodies 2 during their reading.

In the tapered portion 21, the scanbody 2 has a groove 23 which surrounds the tapered portion 21 over 360° and accommodates a part of the thread ring 3.

The thread ring 3 is an elastic component which has a figure-of-eight shape and is constituted by two annular portions which are joined radially and are arranged so that the respective axes are rotated at 90° to each other.

A first annular portion 31 is arranged in the groove 23 of the scanbody 2, as described above, allowing it an always correct positioning.

A second annular portion 32 can be crossed by the linking thread 4 and is oriented on the palatal or lingual side of the scanbody 2.

In order to allow the scanner a more fluid and regular reading, each elastic ring 3 is colored differently from the others used in the scan.

The linking thread 4 passes, with a predetermined friction, through the annular portions 32 of each thread ring 3, consequently rendering the reading fluid and uniform, by the intraoral scanner.

The linking thread 4 is a thread made of plastic material which can be cut so as to be of the length that is suitable for the individual clinical-prosthetic case and has variable cross-sections.

In order to allow a more fluid and regular reading to the scanner, the linking thread 4 is characterized by segments of different color.

The reverse or “inverted” scan, according to the present invention, does not occur inside the oral cavity but externally, with the consequent reduction of stress on the chair, both for the patient and for the professional.

This scan, therefore, can be entrusted even to the prosthodontist, since one only has to read the manufactured article, by means of the laboratory scanner, and one does not need to interact directly with the patient.

Therefore, in the technique with reverse scan, instead of reading the gum parables in the oral cavity once the prosthesis has been removed, the scanner reads all the morphological anatomical characteristics directly from the provisional implant, including the subgingival component.

In this manner, one avoids reading any modifications of the soft tissues which, as mentioned, due to their natural elasticity, during prosthetic maneuvers can undergo unwanted morphological changes with the traditional intraoral scan method.

The reverse scan defines negative volumes which, by means of specific software, are processed and rendered positive, allowing correct prosthetic processing.

The reverse method also allows to perform a reading not only by means of intraoral scanners (a video camera which can be oriented manually and is dependent on an operator, which process the scan by means of sequences of images) but also so-called laboratory scanners, which, being able to accommodate therein the entire prosthesis and the corresponding components of the present invention, allow a single, more precise scan in a single time.

The scan is therefore more defined and systematic.

The match between the traditional scan and the reverse one, according to the present invention, also allows to perform a passiveness control on the files, which is very efficient during design and extremely useful to increase the level of reliability and passiveness of the prosthetic article.

FIGS. 8-19 show the apparatus, used for intraoral scans, generally designated by the reference numeral 101, which includes a plurality of scanbodies 102, each of which is surrounded by a thread ring 3, which in turn engages a portion of a linking thread 4.

The scanbody 102 has a substantially cylindrical shape and includes a first hexagonal portion with double conicity 124.

The first hexagonal portion with double conicity 124 has one of the six sides with a smooth face 125.

The scanbody 102 also has a second hexagonal portion 126, which is joined to the first hexagonal portion 124 and is provided with protrusions 122.

The second hexagonal portion 126 also has a groove 123, which surrounds the second hexagonal portion 126 through 360° and accommodates a part of the thread ring 3.

The double conicity of the first hexagonal portion 124 increases the reading surface of the scanner while the smooth face 125 provides identification.

Each scanbody 102 is rendered better recognizable by the scanner by applying three dimensionally a specific series of variable protrusions 122, for example of three different types, arranged on the second hexagonal portion 126 at the smooth face.

In this manner, by reading the various detected protrusions, the scanner is able to avoid confusing the scanbodies 102 observed during reading since they are unique.

One of the two rings of elastic thread 3 is arranged in the dedicated groove 123 of the scanbody 102, allowing an always correct positioning, the other ring being crossed by the linking thread 4 and oriented on the palatal or lingual side of the scanbody 102.

The linking thread 4 passes with predefined friction through the rings of the individual thread rings 3 and renders a fluid and uniform reading by the intraoral scanner.

The linking thread 4 is a thread made of plastic material which can be sectioned in order to be of the length suitable for the individual clinical-prosthetic case and has variable cross-sections.

The apparatus according to the present invention, as exemplified in the embodiment shown in FIGS. 13-19, allows to enhance the reading and precision capabilities of the intraoral scanner, preventing incorrect positional interpretations of the individual scanbodies and also preventing distortion phenomena due to an excessive distance between the scanbodies and the curvature of the arch being scanned.

According to a further aspect of the invention, the apparatus is provided with a device adapted to minimize the distortion of the arches during the acquisition of the scan.

The device includes modified thread rings which are adapted to act as a bridge between the two sides of the arch.

A modified thread ring, designated by the reference numeral 203 in FIGS. 20 and 21, has a first ring 231 through which a linking thread 4 passes and to which a first band 233 is applied.

A second ring 232, through which a linking thread 4 passes, has a second band 234. When the bands 233 and 234 of the two rings 231 and 232 meet on a central line, they are fixed to each other by virtue of fixing means constituted by holes 235, formed in the first band 233, and pins 236, formed in the second band 234, according to the male/female principle.

The purpose of this bridge-like band is to offer the scanner a continuity in reading between the two sides of the arches, creating a continuous flow scan which is shaped like a triangle and therefore to reduce to a minimum any distortions.

In practice it has been found that the invention achieves the intended aim and objects, a scanning system being provided which allows to overcome the drawbacks of the prior art.

The system according to the present invention allows to enhance the reading and precision capabilities of the intraoral scanner, preventing incorrect interpretations of the position of the individual scanbodies and also preventing distortion phenomena caused by an excessive distance between the scanbodies and by the curvature of the scanned arch.

The system according to the present invention also allows to design and manufacture prosthetic articles that are extremely precise and therefore biomechanically passive, consequently facilitating a prosthetic result that is better more stable over time. The present invention is also advantageous to the operator, by allowing a faster, and at the same time more reliable, digital impression acquisition.

The materials used, as well as the dimensions, may of course be any according to the requirements and the state of the art.

This application claims the priority of Italian Patent Application No. 102020000031670, filed on 21 Dec. 2020, the subject matter of which is incorporated herein by reference.

1-12. (canceled)

13. An apparatus for digitally scanning dental prosthetic implants, comprising a plurality of scanbodies, each surrounded by a thread ring; each said thread ring engaging a portion of a linking thread; each said scanbody being associable with a prosthesis or with the oral cavity.

14. The apparatus according to claim 13, wherein said reverse scanbody has a tapered portion adapted to engage a prosthesis.

15. The apparatus according to claim 13, wherein said scanbody is flattened in a direction of the buccal vestibules and has two regular faces adapted to be recognized by a scanner.

16. The apparatus according to claim 13, wherein said scanbody comprises facets and protrusions on a vestibular side, said facets and protrusions identifying said scanbody in order to differentiate it from the other said scanbodies.

17. The apparatus according to claim 13, wherein said tapered portion of said scanbody has a groove which surrounds said tapered portion through 360° and accommodates part of said thread ring.

18. The apparatus according to claim 13, wherein said thread ring is an elastic component having a figure-of-eight shape and is constituted by two annular portions which are radially joined and arranged with their respective axes rotated at 90° to each other.

19. The apparatus according to claim 13, wherein said tapered portion of said scanbody comprises a groove which surrounds said tapered portion through 360°; said first annular portion being arranged in said groove.

20. The apparatus according to claim 13, wherein it comprises a second annular portion which can be crossed by said linking thread and is directed toward a palatal or lingual side of said scanbody.

21. The apparatus according to claim 13, wherein each one of said elastic rings is colored differently with respect to the other ones used in the scan.

22. The apparatus according to claim 13, wherein each one of said scanbodies has a substantially cylindrical shape and comprises a first hexagonal portion with a double conicity having at least one smooth face; said scanbody further comprising a second hexagonal portion which is joined to said first hexagonal portion and is provided with protrusions; said second hexagonal portion further having a groove; said groove surrounding said second hexagonal portion through 360°.

23. The apparatus according to claim 13, wherein said scanbody comprises protrusions arranged on said second hexagonal portion at said smooth face.

24. A method for digitally scanning dental prosthetic implants according to claim 13, comprising: removing dental prostheses from the oral cavity of a patient; for performing a scan outside said oral cavity, associating each scanbody with a respective prosthesis; associating said scanbodies with one another by means of said thread rings and said linking thread, forming a unit; performing a digital scan of said unit; for performing a scan in said oral cavity, screwing each said scanbody with an implant inserted in the bone in said oral cavity; associating said scanbodies with one another by means of said thread rings and said linking thread, forming a unit; performing a digital scan of said unit in said oral cavity.
